(19) United States
(12) Patent Application Publication
(10) Pub. No.: US 2021/0330197 A1
(43) Pub. Date: Oct. 28, 2021

(54) BIOLOGICAL INFORMATION DETECTION DEVICE

(71) Applicant: DENSO CORPORATION, Kariya-city (JP)

(72) Inventors: Shunsuke SHIBATA, Nisshin-city (JP); Takashi SAITOU, Nisshin-city (JP); Koichi YAMADA, Kariya-city (JP)

(21) Appl. No.: 17/368,389

(22) Filed: Jul. 6, 2021

Related U.S. Application Data
(63) Continuation of application No. PCT/JP2019/045665, filed on Nov. 21, 2019.

(30) Foreign Application Priority Data
Jan. 10, 2019 (JP) 2019-002912

(51) Int. Cl. A61B 5/0245; A61B 5/18

(52) U.S. Cl. CPC A61B 5/0245; A61B 2562/0219; A61B 5/18

(57) ABSTRACT

In a biological information detection device, a frequency characteristic indicating a relation between a frequency and an intensity is acquired with respect to each of a plurality of biological signals input respectively from a plurality of biological activity sensors arranged at a plurality of positions different from each other to detect a biological activity of a person. A synthetic frequency characteristic indicating the relation between the frequency and the intensity is obtained by synthesizing a plurality of frequency characteristics acquired from the plurality of biological signals. Biological information on the biological activity is calculated based on the synthetic frequency characteristic.

### CROSS REFERENCE TO RELATED APPLICATIONS

The present application is a continuation application of International Patent Application No. PCT/JP2019/045665 filed on Nov. 21, 2019, which designated the U.S. and claims the benefit of priority from Japanese Patent Application No. 2019-002912 filed on Jan. 10, 2019. The entire disclosures of all of the above applications are incorporated herein by reference.

### TECHNICAL FIELD

The present disclosure relates to a biological information detection device.

### BACKGROUND

There is described a technology that subtracts a time waveform of the signal detected by a first piezoelectric element arranged near the seat mounting bracket from a time waveform of the signal detected by a second piezoelectric element embedded in the part of the backrest of the seat near the occupant's heart. Such a technology can remove vehicle noise included in the biological signal detected by the second piezoelectric element. Then, the technology calculates the heart rate of the passenger from the biological signal from which the vehicle noise is removed.

### SUMMARY

According to an example of the present disclosure, a biological information detection device is provided as follows. That is, a frequency characteristic indicating a relation between a frequency and an intensity is acquired with respect to each of a plurality of biological signals input respectively from a plurality of biological activity sensors arranged at a plurality of positions different from each other to detect a biological activity of a person. A synthetic frequency characteristic indicating the relation between the frequency and the intensity is obtained by synthesizing a plurality of frequency characteristics acquired from the plurality of biological signals. Biological information on the biological activity is calculated based on the synthetic frequency characteristic.

### BRIEF DESCRIPTION OF THE DRAWINGS

The objects, features, and advantages of the present disclosure will become more apparent from the following detailed description made with reference to the accompanying drawings. In the drawings:

FIG. 1 is an overall configuration diagram of a biological information detection system;

FIG. 2 is a flowchart of processing executed by a processing unit;

FIG. 3 is a diagram illustrating signal conversion and signal synthesis;

FIG. 4 is a flowchart of processing executed by a processing unit in a second embodiment;

FIG. 5 is a diagram illustrating a weight calculation process;

FIG. 6 is a flowchart of processing executed by a processing unit in a third embodiment;

FIG. 7 is a flowchart of processing executed by a processing unit in a fourth embodiment;

FIG. 8 is an overall configuration diagram of a biological information detection system according to a fifth embodiment;

FIG. 9 is a flowchart of processing executed by a processing unit; and

FIG. 10 is a flowchart of a biological information detection system according to a sixth embodiment.

### DETAILED DESCRIPTION

### First Embodiment

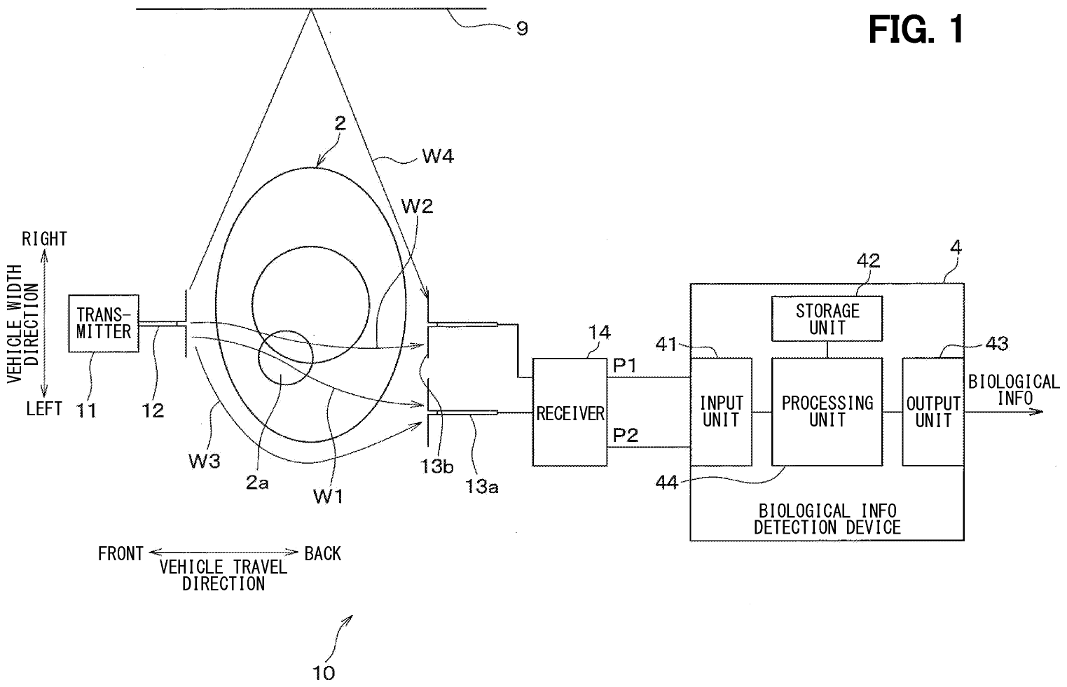

Hereinafter, a first embodiment will be described. As shown in FIG. 1, a biometric information detection system according to the present embodiment, which is mounted on a vehicle, calculates and outputs the heart rate of a person 2 seated in the driver's seat of the vehicle as biometric information. The biological information of the person 2 means the information related to the biological activity of the person 2. This biometric information detection system includes a biometric information detection device 4, a transmitter 11, a transmitting antenna 12, a first receiving antenna 13a, a second receiving antenna 13b, and a receiver 14.

The transmitter 11 outputs a transmission signal having a predetermined frequency (for example, a frequency in the 900 MHz band) to the transmitting antenna 12. The transmitting antenna 12 is arranged on the front side of the instrument panel in the vehicle interior in the vehicle traveling direction with respect to the driver's seat. The transmitting antenna 12 transmits a radio wave signal corresponding to the transmission signal from the transmitter 11 toward the upper body of the person 2 seated in the driver's seat.

A first receiving antenna 13a and a second receiving antenna 13b are arranged to face the transmitting antenna 12 such that the person 2 and the driver's seat are sandwiched between (i) the transmitting antenna 12 and (ii) both the first receiving antenna 13a and the second receiving antenna 13b. Specifically, the first receiving antenna 13a and the second receiving antenna 13b are arranged at different positions in the vehicle width direction. For example, the first receiving antenna 13a and the second receiving antenna 13b may be embedded in the seat back of the vehicle. The first receiving antenna 13a and the second receiving antenna 13b are configured to be able to receive the radio wave signal transmitted from the transmitting antenna 12. The first receiving antenna 13a and the second receiving antenna 13b each correspond to a biological activity sensor.

The receiver 14 amplifies and outputs the radio wave signal received by the first receiving antenna 13a and the second receiving antenna 13b. Specifically, the receiver 14 amplifies the radio wave signal received by the first receiving antenna 13a and outputs it as the biological signal P1 to the biological information detection device 4. Further, the receiver 14 amplifies the radio wave signal received by the second receiving antenna 13b and outputs it as the biological signal P2 to the biological information detection device 4.

The biological information detection device 4 includes an input unit 41, a storage unit 42, an output unit 43, and a processing unit 44. The input unit 41 outputs the biological signals P1 and P2, which are analog signals input from the receiver 14, to the processing unit 44 as digital signals. The storage unit 42 includes a RAM, a ROM, a writable non-volatile storage medium, and the like. The RAM, ROM, and writable non-volatile storage medium are all non-transitory tangible storage media. The output unit 43 outputs the signal input from the processing unit 44 to an external device outside of the biological information detection device 4. The external device as the output destination may be, for example, an in-vehicle navigation device that provides route guidance or the like, an in-vehicle data communication module that communicates with the outside of the vehicle, or a mobile communication terminal carried by the person 2.

The processing unit 44 is a device that executes processing according to a program recorded in the ROM of the storage unit 42 or the writable non-volatile storage medium, and uses the RAM of the storage unit 42 as a work area at the time of execution.

Hereinafter, the operation of the biometric information detection system having the above configuration will be described. The transmitter 11 outputs a transmission signal having a predetermined frequency to the transmitting antenna 12. Then, the transmitting antenna 12 transmits a radio wave signal corresponding to the transmission signal from the transmitter 11 toward the driver's seat and the person 2.

A part of this radio wave signal, which passes through the body of the person 2, is received by the first receiving antenna 13a and the second receiving antenna 13b. The body of the person 2 functions as a dielectric with respect to the radio wave signal. Therefore, when the radio wave signal is transmitted through the body of the person 2, a dielectric loss occurs in the electric field intensity of the radio wave signal. The shape of the heart 2a changes as it expands and contracts. The radio wave signals W1 and W2 pass through the heart 2a as shown in FIG. 1 and reach the first receiving antenna 13a and the second receiving antenna 13b, respectively. In such radio wave signals W1 and W2, the dielectric loss that occurs in the electric field intensity changes according to the heart rate of the heart 2a.

The intensity of the radio wave signal received by each of the first receiving antenna 13a and the second receiving antenna 13b thereby includes a component that changes in synchronization with the heart rate according to the heart rate of the heart 2a. Therefore, the level of the electric signals and the biological signals P1 and P2 output from each of the first receiving antenna 13a and the second receiving antenna 13b to the receiver 14 by receiving the radio wave signal include a component that fluctuates in synchronization with the heart rate according to the heart rate of the heart 2a.

On the other hand, the radio wave signals from the transmitting antenna 12 include the radio wave signal that does not pass through the body of the person 2 such as a diffracted wave W3 and a reflected wave W4 as shown in FIG. 1. Such a diffracted wave W3 or a reflected wave W4 may be received as a radio wave signal by only one of the first receiving antenna 13a and the second receiving antenna 13b. The diffracted wave W3 is a radio wave signal that goes around the left side of the person 2. The reflected wave W4 is a radio wave signal reflected by the door 9 on the right side of the person 2.

These diffracted waves W3 and reflected waves W4 include not only signals necessary for calculating the biological information of the person 2, but also noise caused by vibration caused by the running of the vehicle, noise caused by disturbance from the outside of the vehicle, and the like. Therefore, the radio wave signal received by the first receiving antenna 13a and the radio wave signal received by the second receiving antenna 13b are different from each other in the type and property of the contained noise. In a sense, the noise component appears randomly at each measurement point. This is because the positions of the first receiving antenna 13a and the second receiving antenna 13b are different from each other.

When the radio wave signals are received in this way, the first receiving antenna 13a and the second receiving antenna 13b each output a reception signal whose signal intensity changes depending on the electric field intensity of the received radio wave signal. The receiver 14 outputs the biological signal P1 in which the received signal input from the first receiving antenna 13a is amplified to the biological information detection device 4. Further, the receiver 14 outputs the biological signal P2 in which the received signal input from the second receiving antenna 13b is amplified to the biological information detection device 4.

As described above, the transmitter 11, the transmitting antenna 12, the first receiving antenna 13a, the second receiving antenna 13b, and the receiver 14 continuously operate. As a result, the biological signals P1 and P2 whose signal intensity changes with the passage of time are continuously input to the input unit 41 of the biological information detection device 4. Each of the biological signals P1 and P2 includes (i) a signal component representing the heart rate, which is biological information, and (ii) noise unrelated to the biological information. The biological signal P1 and the biological signal P2 are different from each other in type and property of the contained noise.

As described above, the input unit 41 outputs a digital signal having a value corresponding to the signal intensity of the input biological signals P1 and P2 to the processing unit 44. Therefore, information on the intensity change of the biological signals P1 and P2 with the time course is input to the processing unit 44. The information on the intensity change of the biological signals P1 and P2 with the time course is a time waveform, that is, a waveform in the time domain. More specifically, this time waveform contains information on the signal intensity at each of a plurality of discrete sampling timings separated by a predetermined time interval.

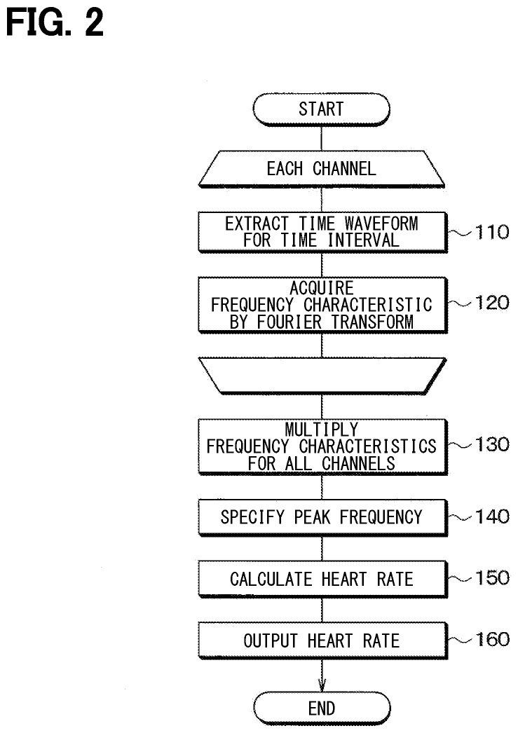

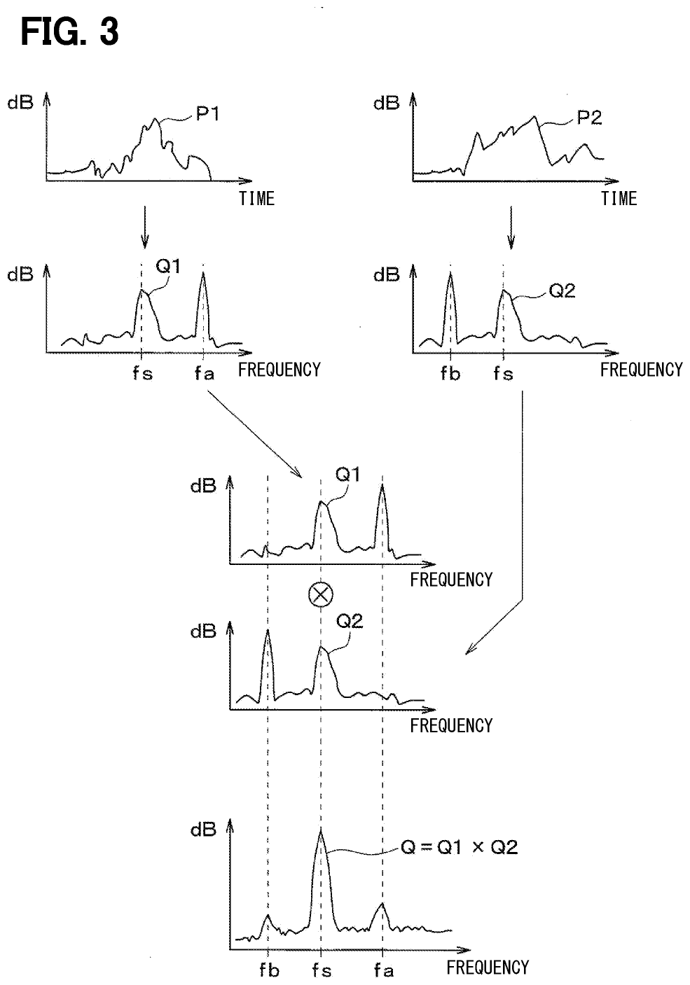

The processing unit 44 executes the process shown in FIG. 2 by reading and executing a predetermined program from the ROM of the storage unit 42 or the writable non-volatile storage medium. FIG. 3 illustrates a state of signal conversion realized by this process.

The processing unit 44 calculates the heart rate of the person 2 based on the time waveforms of the biological signals P1 and P2 by the process of FIG. 2. Specifically, the processing unit 44 first performs the processing of steps 110 and 120 once for each channel, for a total of the number of channels. Here, one channel is assigned to each of receiving antennas. That is, the first receiving antenna 13a is assigned with a first channel, and the second receiving antenna 13b is assigned with a second channel.

In step 110 corresponding to the first channel, the processing unit 44 extracts the time waveform of the input biological signal P1 for a time interval with a predetermined length. For example, only the time interval from one second before to the present time is extracted. Subsequently, in step 120 corresponding to the first channel, the processing unit 44 performs a discrete Fourier transform on the time waveform extracted in the immediately preceding step 110. As a result, the frequency characteristic Q1 indicating the relation between the frequency and the intensity of the biological signal P1 in the time interval is acquired. Frequency characteristics are waveforms in the frequency domain.

In step 110 corresponding to the second channel, the processing unit 44 extracts the time waveform of the input biological signal P2 for the above-mentioned time interval. Subsequently, in step 120 corresponding to the second channel, the processing unit 44 performs a discrete Fourier transform on the time waveform extracted in the immediately preceding step 110. As a result, the frequency characteristic Q2 indicating the relation between the frequency and the intensity of the biological signal P2 in the time interval is acquired.

In this way, the processing unit 44 calculates the frequency characteristics Q1 and Q2 in the same frequency range in the plurality of channels from the time waveforms of the biological signals P1 and P2 in the same time interval in the plurality of channels. The frequency waveform thus obtained in step 120 of each channel contains information on the signal intensity at each of a plurality of discrete frequencies separated by a predetermined frequency interval in more detail.

As shown in FIG. 3, these frequency characteristics Q1 and Q2 have a plurality of peaks. Here, the peak means that the intensity is equal to or higher than a predetermined value and is maximized. These peaks include a peak derived from the pulse of the heart 2a and a peak derived from other noise.

In the example of FIG. 3, in the frequency characteristic Q1, the peak at the frequency fs is a peak derived from the pulse of the heart 2a, and the peak at the frequency fa is a peak derived from the noise included in the diffracted wave W3. Further, in the frequency characteristic Q2, the peak at the frequency fs is a peak derived from the pulse of the heart 2a, and the peak at the frequency fb is a peak derived from the noise included in the reflected wave W4.

As described above, the noise frequency is often different when the position of the receiving antenna is different. This is because when the positions of the plurality of receiving antennas are different from each other, the types and properties of noise received by the plurality of receiving antennas are different. On the other hand, the frequency fs of the peak derived from the heart 2a is likely to be the same for the biological signal from any receiving antenna.

Assume that the pulse rate of the heart 2a is calculated based only on the frequency characteristic Q1. In this assumption, when the intensity of the peak of the frequency fa is higher than the intensity of the peak of the frequency fs, there is a high possibility that the pulse rate of the heart 2a is calculated based on the frequency fa derived from the noise. Also, assume that the pulse rate of the heart 2a is calculated based only on the frequency characteristic Q2. In this assumption, when the intensity of the peak of the frequency fb is higher than the intensity of the peak of the frequency fs, there is a high possibility that the pulse rate of the heart 2a will be calculated based on the frequency fb derived from the noise. In the present embodiment, as will be described later, the pulse rate is calculated by utilizing the synthesis of the frequency characteristic Q1 and the frequency characteristic Q2 in the frequency domain.

After the repetition by the number of processing channels in steps 110 and 120 is completed, the processing unit 44 proceeds to step 130. In step 130, the frequency characteristics obtained in steps 110 and 120 for all channels, that is, the frequency characteristics Q1 of the biological signal P1 and the frequency characteristics Q2 of the biological signal P2 are multiplied by each other. Then, the relation between the frequency and the intensity obtained as a result of the multiplication is defined as a synthetic frequency characteristic Q. This multiplication corresponds to a synthesis.

Specifically, the synthetic frequency characteristic Q (vi) is obtained by the equation Q (vi)=Q1 (vi)×Q2 (vi). Here, Q1 (vi) is an expression of the frequency characteristic Q1 as a function of the above-mentioned discrete plurality of frequencies vi (where i=1, 2, . . . n, n is the total number of the discrete plurality of frequencies). Further, Q2 (vi) is an expression of the frequency characteristic Q2 as a function of the discrete plurality of frequencies vi (where i=1, 2, . . . n, n is the total number of the discrete plurality of frequencies). That is, the synthetic frequency characteristic Q is obtained by multiplying the frequency characteristic Q1 and the frequency characteristic Q2 with respect to each same frequency in the frequency domain.

In the synthetic frequency characteristic Q thus obtained, the intensity of a peak appearing in only part of the frequency characteristics Q1 and Q2 used in the synthesis is weakened by this synthesis. On the other hand, in the synthetic frequency characteristic Q, a peak appearing in all of the frequency characteristics Q1 and Q2 used in the synthesis is strengthened by this synthesis. As a result, as shown in FIG. 1, the peak of the frequency fs derived from the heart rate of the heart 2a becomes the peak having the highest intensity.

Subsequently, in step 140, the processing unit 44 specifies the frequency of the peak having the maximum intensity among the peaks of the synthetic frequency characteristic Q obtained in the immediately preceding step 130, that is, the peak frequency. In the example of FIG. 3, the frequency fs is specified as the peak frequency.

Subsequently, in step 150, the heart rate of the heart 2a is specified based on the peak frequency specified in the immediately preceding step 140. For example, if the peak frequency is 1 Hz, the heart rate will be 60 beats/minute as a result of multiplying it by 60.

Subsequently, in step 160, the processing unit 44 outputs the heart rate calculated in the immediately preceding step 150 to the output unit 43 as a digital data. The output unit 43 outputs the digital data of the heart rate input from the processing unit 44 in this way to an external device outside of the biological information detection device 4.

As described above, the biometric information, which is information related to biological activity, is calculated based on the synthetic frequency characteristic Q obtained by synthesizing a plurality of frequency characteristics Q1 and Q2. The present inventor has focused on the fact that the frequency characteristics of non-noise heart rate-derived components of biological signals are generally stable. Looking at the biological signal in the time domain, if the noise component and the heart rate-derived component are received at different positions, the waveform will be significantly different. However, in the frequency domain, the heart rate-derived component almost always peaks at a frequency corresponding to the heart rate regardless of the position of the biological activity sensor. On the other hand, the peak frequency of the noise component differs greatly depending on the position of the biological activity sensor even when viewed in the frequency domain.

The inventor found that if biological signals are detected by the first receiving antenna 13a and the second receiving antenna 13b arranged at different positions, the frequency characteristics of the noise contained in the biological signals detected by those antennas tend to be significantly different from each other. The inventor came up with the idea of using it.

That is, as described above, the inventor came up with an idea that the frequency characteristics Q1 and Q2 of the biological signals P1 and P2 from the first receiving antenna 13a and the second receiving antenna 13b arranged at different positions are synthesized in the frequency domain instead of the time domain. As a result, the non-noise frequency portions of the biological signals P1 and P2 are strengthened, and the noise frequency portions are not strengthened. Therefore, in the synthetic frequency characteristic Q obtained by synthesis, the influence of noise is suppressed.

Moreover, since the frequency characteristics Q1 and Q2 are synthesized in the frequency domain, the phase shift does not affect the noise suppression. There may be a gap between the time it takes for the radio wave signal W1 to reach the first receiving antenna 13a from the transmitter 11 and the time it takes for the radio wave signal W2 to reach the second receiving antenna 13b from the transmitter 11. In this case, a phase shift occurs between the biological signal P1 and the biological signal P2 input to the input unit 41. If the biological signals P1 and P2 are synthesized in the time domain, the synthesis is performed with this deviation remaining, or a process for correcting the phase shift is required. In the former case, the accuracy of heart rate calculation is reduced. In the latter case, the extra processing load increases. On the other hand, since the frequency characteristics Q1 and Q2 show the intensity distribution in the frequency domain, they are not easily affected by the phase shift, so that the above effects can be obtained.

Further, the processing unit 44 obtains the synthetic frequency characteristic Q by multiplying the frequency characteristics Q1 and Q2 by each other. In this way, the S/N ratio of the synthetic frequency characteristic is improved by obtaining the synthetic frequency characteristic by multiplying the plurality of frequency characteristics Q1 and Q2 by each other. For example, addition can be considered as synthesis other than multiplication; however, in the case of addition, the effect of strengthening the peaks corresponding to the heart rate by synthesis is lower than in the case of multiplication.

Further, the processing unit 44 synthesizes the frequency characteristics Q1 and Q2 at the same frequency. As a result, the S/N ratio of the synthetic frequency characteristic is improved. The frequency characteristics Q1 and Q2 may be multiplied with a slight frequency shift. However, in this case, the effect of strengthening the peaks corresponding to the heart rate by synthesis is reduced as compared with the case of synthesizing the same frequencies.

The autonomous driving system of NHTSA index level 3 or lower operates while the driver monitors the driving of the vehicle, and the driver is responsible for driving. NHTSA is an abbreviation for National Highway Traffic Safety Administration.

On the other hand, it has been reported in many academic societies that the automatic driving system reduces the psychological burden on the driver and reduces the alertness. Therefore, in recent years, the development of a system that detects the alertness of the driver and displays a warning or the like according to the result has been studied. Biological information such as the driver's heart rate and respiratory rate is often used as information for detecting the driver's alertness.

The sensor used to acquire these biological information is usually mainly attached to a finger or the like. However, when the target is a driver, the non-contact type sensor is advantageous because of the demands such as “does not interfere with driving” and “constant measurement is required”. The non-contact sensor does not need to be in constant contact with the driver even if it needs to be constantly measured. In contrast, non-contact sensors are also available.

Such non-contact type sensor includes a radio wave type sensor as in the present embodiment. Therefore, the heart rate output from the biological information detection device 4 of the present embodiment may be output to the alertness detection device that detects the alertness of the driver.

Since the non-contact type sensor is a non-contact type, the S/N tends to decrease due to an external noise component. As a method for removing the noise component, there is a method as described in Patent Literature 1, but it may be difficult to remove noise in a frequency band near the heart rate due to a phase shift. Since the biological information detection device 4 of the present embodiment synthesizes the frequency characteristics in the frequency domain, it is more robust against the phase shift than a known method.

In the present embodiment, the processing unit 44 functions as a characteristic acquisition unit by executing step 120, functions as a synthesis unit by executing step 130, and functions as a calculation unit by executing step 150.

### Second Embodiment

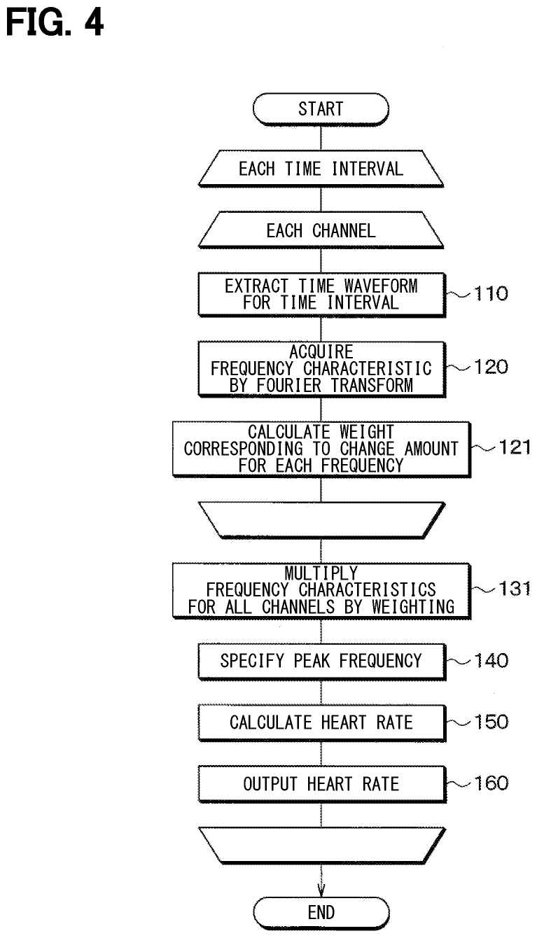

Next, a second embodiment will be described focusing on differences from the first embodiment. In the present embodiment, the process executed by the processing unit 44 is replaced with the process of FIG. 4 with respect to the first embodiment. Other than that, the configuration and operation of the present embodiment are the same as those of the first embodiment.

Hereinafter, the contents of the process of FIG. 4 will be described. The steps with the same reference numerals in FIGS. 2 and 4 are the same except for the parts described below.

In the process of FIG. 4, the processing unit 44 first performs (i) the processing for each channel and (ii) the processing of steps 131, 140, 150, and 160, in this order, each time a time interval with a predetermined length (for example, 1 second) elapses. In the processing for each channel, the processing of steps 110, 120, and 121 is performed once for each channel, for a total of the number of channels.

The processing for each channel and the processing in steps 131, 140, 150, and 160 each time interval has elapsed will be described later.

First, the processing for each channel will be described. The processing of steps 110, 120, and 121 for each channel is as follows. In step 110, the processing unit 44 extracts the time waveform of the biological signal of the channel input from the input unit 41 in the time interval. Subsequently, in step 120, the time waveform extracted in the immediately preceding step 110 is subjected to a discrete Fourier transform to acquire a frequency characteristic indicating the relation between the frequency and the intensity of the biological signal in the time interval for the channel.

Subsequently, in step 121, the weight ω corresponding to the time-course change amount for each frequency in the frequency characteristic of the biological signal of the channel in the time interval is calculated. This processing will be specifically described below.

First, the processing unit 44 calculates the time-course change amount in intensity for each frequency in the frequency characteristic of the channel. This calculation is performed based on (i) the frequency characteristic calculated in step 120 immediately before in the present time interval and (ii) the frequency characteristic calculated in step 120 for the same channel in the previous time interval immediately before the present time interval. Note that if step 121 this time is the execution opportunity of the step 121 first for the same channel, the time-course change amount in intensity for each frequency is set to zero regardless of the frequency.

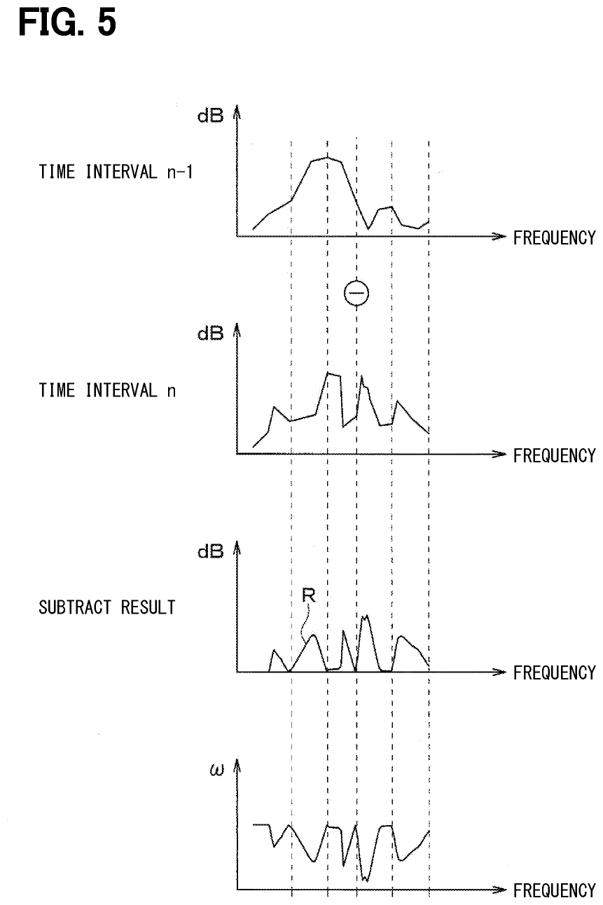

For example, as illustrated in FIG. 5, the processing unit 44 subtracts, at the same frequency, the frequency characteristic of the biological signal in the (n−1)-th time interval of a specific channel from the frequency characteristics of the biological signal in the n-th time interval of the specific channel. Here, n is a natural number. Then, the processing unit 44 calculates the absolute value of the subtraction result, and sets the absolute value as a time-course change amount R for each frequency of the intensity as shown in FIG. 5. The n-th time interval is the time interval newly passed this time; the (n−1)-th time interval is the time interval one time before the time interval newly passed this time.

Then, the processing unit 44 calculates the weight ω for each frequency as an amount that becomes smaller as the time-course change amount R becomes larger, as shown in FIG. 5, based on the time-course change amount R in the intensity calculated in this way for each frequency. The weight calculated in this way is a weight corresponding to the frequency characteristic of the biological signal of the channel in the time interval. The value of the weight ω is always 0 or positive. Thus, the processing in step 121 is completed.

Such processing of steps 110, 120, 121 is performed for each of the possible combinations of the plurality of channels. Therefore, the weight ω for each frequency corresponding to the frequency characteristic of the biological signal of each channel is calculated.

After the repetition by the number of processing channels of steps 110, 120, and 121 is completed, the processing unit 44 proceeds to step 131. In step 131, the frequency characteristics obtained in step 120 for all channels are multiplied by the weighting of the weight ω obtained in step 121 for all channels. Then, the relation between the frequency and the intensity obtained as a result of the weighted multiplication is defined as the synthetic frequency characteristic Q. This multiplication corresponds to a synthesis.

Specifically, the synthetic frequency characteristic Q (vi) is obtained by the formula

Q (vi)=ω1(vi)×Q1(vi)×ω2(vi)×Q2(vi).

Here, Q1 (vi) is a frequency characteristic of the biological signal of the channel corresponding to the first receiving antenna 13a in the n-th time interval. Further, Q2 (vi) is a frequency characteristic of the biological signal of the channel corresponding to the second receiving antenna 13b in the n-th time interval. Further, ω1 (vi) is a weight ω for each frequency vi of the biological signal of the channel corresponding to the first receiving antenna 13a in the n-th time interval. Further, ω2 (vi) is a weight ω for each frequency vi of the biological signal of the channel corresponding to the second receiving antenna 13b in the n-th time interval.

That is, the synthetic frequency characteristic Q is obtained by multiplying the frequency characteristic Q1, the frequency characteristic Q2, the weight ω1, and the weight ω2 in the same n-th time interval each the same frequency in the frequency domain.

In this way, synthesis that reflects the frequency-dependent weight ω is performed. Therefore, at the frequency where the value of the weight ω is small, the value of the synthetic frequency characteristic Q is suppressed. At the frequency where the value of the weight ω is large, the value of the synthetic frequency characteristic Q is emphasized.

Subsequently, in step 140, the processing unit 44 specifies the peak frequency of the synthetic frequency characteristic Q obtained in the immediately preceding step 131, as in the first embodiment. Subsequently, in step 150, the heart rate of the heart 2a is specified based on the peak frequency specified in the immediately preceding step 140, as in the first embodiment. Subsequently, in step 160, the heart rate calculated in the immediately preceding step 150 is output to the output unit 43 as digital data. The output unit 43 outputs the digital data of the heart rate input from the processing unit 44 in this way to an external device outside of the biological information detection device 4.

As a result, the same effects as those of the first embodiment can be obtained. In addition, the processing unit 44 calculates the time-course change amount in intensity for each frequency with respect to each of the frequency characteristics of biological signals of multiple channels in a predetermined time interval, based on the frequency characteristics of the biological signals of the same channel in the time interval immediately before that time interval. Then, the processing unit 44 calculates the weight ω for each frequency according to the time-course change amount. Then, the processing unit 44 synthesizes the frequency characteristics of the plurality of channels in a state in which the plurality of weights w corresponding to the plurality of channels are reflected, and obtains the synthetic frequency characteristic Q.

As described above, the frequency characteristics of noise tend to differ greatly depending on the installation locations of the first receiving antenna 13a and the second receiving antenna 13b. Further, not only that, they tend to differ greatly depending on the difference in the acquisition period of the biological signal. On the other hand, the frequency characteristics of the components of the biological signals that reflect the heart rate rather than the noise are generally stable over time. Focusing on these points, the inventor came up with the idea that frequencies whose intensities fluctuate significantly over time are considered to be derived from noise.

For that purpose, as described above, the processing unit 44 reflects the weight for each frequency in the synthetic frequency characteristic according to the time-course change amount in intensity for each frequency based on the frequency characteristic in the period other than the predetermined time interval. Thereby, the S/N ratio of the synthetic frequency characteristic can be further improved by utilizing the characteristic of the biological signal in the frequency domain.

Further, each of the plurality of calculated weights w becomes smaller as the absolute value of the corresponding time-course change amount at the same frequency is larger. Here, the corresponding time-course change amount means the time-course change amount used to calculate the weight ω. By doing so, the weight ω can be set as a more intuitive quantity.

In the present embodiment, the processing unit 44 functions as a characteristic acquisition unit by executing step 120, functions as a synthesis unit by executing step 131, and functions as a calculation unit by executing step 150. Further, the processing unit 44 functions as a change weight calculation unit by executing step 121.

### Third Embodiment

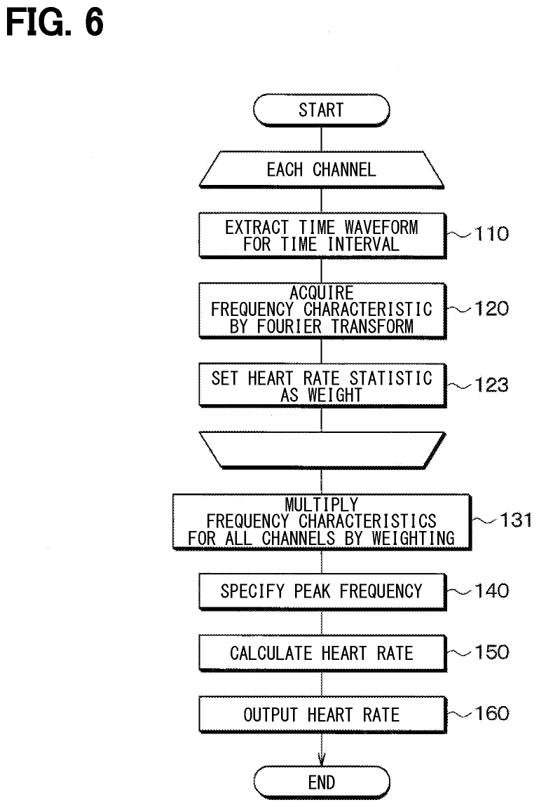

Next, a third embodiment will be described focusing on differences from the first embodiment. In the present embodiment, the process executed by the processing unit 44 is replaced with the process of FIG. 6 with respect to the first embodiment. Other than that, the configuration and operation of the present embodiment are the same as those of the first embodiment.

Hereinafter, the contents of the process of FIG. 4 will be described. The steps with the same reference numerals in FIGS. 2 and 4 are the same except for the parts described below. The processing unit 44 acquires the frequency characteristics for each channel in step 120 in the same manner as in the first embodiment. Then, in step 123, the heart rate statistic is read from the ROM of the storage unit 42 or the writable non-volatile storage medium, and is set as the weight ω. The heart rate statistic has a value for each frequency.

Here, the heart rate statistics will be described. Heart rate varies from person to person. More specifically, the distribution of heart rate in a normal state follows a normal distribution with a fixed mean μ and variance σ. The value for each frequency representing this normal distribution is the heart rate statistic value. The heart rate statistic value is determined in advance by an experiment or the like and recorded in the ROM of the storage unit 42 or a writable non-volatile storage medium.

After the repetition by the number of processing channels of steps 110, 120, and 123 is completed, the processing unit 44 proceeds to step 131. In step 131, the processing unit 44 multiplies the frequency characteristics obtained in step 120 for all channels by the weighting of the weight ω obtained in step 123 for all channels. Then, the relation between the frequency and the intensity obtained as a result of the weighted multiplication is defined as a synthetic frequency characteristic Q. This multiplication corresponds to a synthesis. The weighted multiplication method is the same as in step 131 of the second embodiment.

Subsequently, in step 140, the processing unit 44 specifies the peak frequency of the synthetic frequency characteristic Q obtained in the immediately preceding step 131, as in the first embodiment. Subsequently, in step 150, the heart rate of the heart 2a is specified based on the peak frequency specified in the immediately preceding step 140, as in the first embodiment. Subsequently, in step 160, the heart rate calculated in the immediately preceding step 150 is output to the output unit 43 as digital data. The output unit 43 outputs the digital data of the heart rate input from the processing unit 44 in this way to an external device outside of the biological information detection device 4. As a result, the same effects as those of the first embodiment can be obtained.

Further, the processing unit 44 sets the weight ω as the heart rate statistic value corresponding to the distribution statistic value of the heart rates of a large number of people in normal states for each of the frequency characteristics of the biological signals of the plurality of channels in a predetermined time interval. Then, the processing unit 44 synthesizes the frequency characteristics of the plurality of channels in a state in which the plurality of weights w corresponding to the plurality of channels are reflected, and obtains the synthetic frequency characteristic Q.

In this way, the weight ω corresponding to the heart rate statistic is reflected in the plurality of frequency characteristics obtained by Fourier transforming the plurality of biological signals. By obtaining the synthetic frequency characteristic Q, noise can be stochastically removed.

In the present embodiment, the processing unit 44 functions as a characteristic acquisition unit by executing step 120, functions as a synthesis unit by executing step 131, and functions as a calculation unit by executing step 150.

### Fourth Embodiment

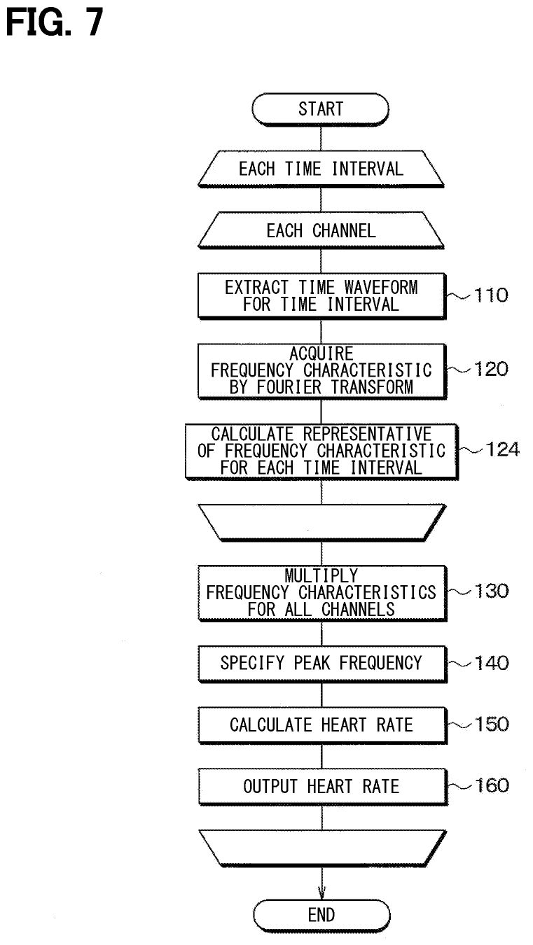

Next, a fourth embodiment will be described focusing on differences from the first embodiment. In the present embodiment, the process executed by the processing unit 44 is replaced with the process of FIG. 7 with respect to the first embodiment. Other than that, the configuration and operation of the present embodiment are the same as those of the first embodiment.

Hereinafter, the contents of the process of FIG. 7 will be described. The steps with the same reference numerals in FIGS. 2 and 7 are the same except for the parts described below.

In the process of FIG. 7, the processing unit 44 first performs the processing for each channel and the processing of steps 130, 140, 150, and 160 in this order each time a time interval with a predetermined length (for example, 1 second) elapses. In the processing for each channel, the processing of steps 110, 120, and 124 are performed once for each channel, for a total of the number of channels.

The processing for each channel and the processing in steps 130, 140, 150, and 160 when each time interval elapses are described later.

First, the processing for each channel will be described. The processing of steps 110, 120, and 124 for each channel is as follows. In step 110, the processing unit 44 extracts the time waveform of the biological signal of the channel input from the input unit 41 in the present time interval. Subsequently, in step 120, the time waveform extracted in the immediately preceding step 110 is subjected to a discrete Fourier transform. By doing so, the frequency characteristic indicating the relation between the frequency and the intensity of the biological signal in the present time interval of the channel is acquired.

Then, in step 124, the representative values of these plurality of frequency characteristics are calculated for each frequency based on the frequency characteristics acquired in step 120 immediately before in the present time interval and the frequency characteristics acquired in step 120 for the same channel in the previous time interval immediately before the present time interval. The representative value means a statistical representative value, for example, an arithmetic mean value, a geometric mean value, or a median value. Moreover, the representative value of a plurality of frequency characteristics is a representative value of the same frequency.

When the present time interval is the first time interval, in step 124, the frequency characteristic itself acquired in the immediately preceding step 120 is used as a representative value.

After the repetition by the number of processing channels of steps 110, 120, and 124 is completed, in step 130, the processing unit 44 multiplies the frequency characteristics obtained in step 124 for all channels by the same frequencies in the same manner as in the first embodiment. Then, the relation between the frequency and the intensity obtained as a result of the multiplication is defined as a synthetic frequency characteristic Q. This multiplication corresponds to a synthesis.

Subsequently, in step 140, the processing unit 44 specifies the peak frequency of the synthetic frequency characteristic Q obtained in the immediately preceding step 130, as in the first embodiment. Subsequently, in step 150, the heart rate of the heart 2a is specified based on the peak frequency specified in the immediately preceding step 140, as in the first embodiment. Subsequently, in step 160, the heart rate calculated in the immediately preceding step 150 is output to the output unit 43 as digital data. The output unit 43 outputs the digital data of the heart rate input from the processing unit 44 in this way to an external device outside of the biological information detection device 4.

As a result, the same effects as those of the first embodiment can be obtained. In addition, the processing unit 44 calculates representative values of a plurality of frequency characteristics acquired for each of two or more time intervals for each of the biological signals of the plurality of channels. Then, the processing unit 44 synthesizes a plurality of calculated representative values of the plurality of channels to obtain the synthetic frequency characteristic Q. In this way, by obtaining the synthetic frequency characteristic using the representative value of two or more time intervals, the S/N ratio of the synthetic frequency characteristic is improved.

In the present embodiment, the processing unit 44 functions as a characteristic acquisition unit by executing steps 120 and 124, functions as a synthesis unit by executing step 130, and functions as a calculation unit by executing step 150.

### Fifth Embodiment

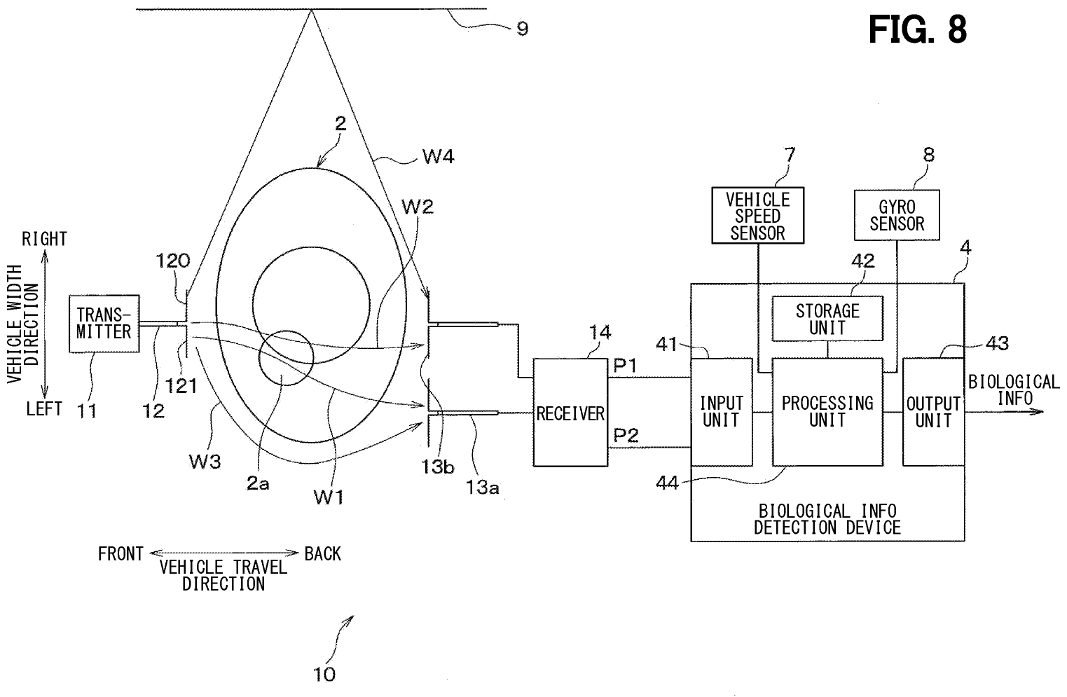

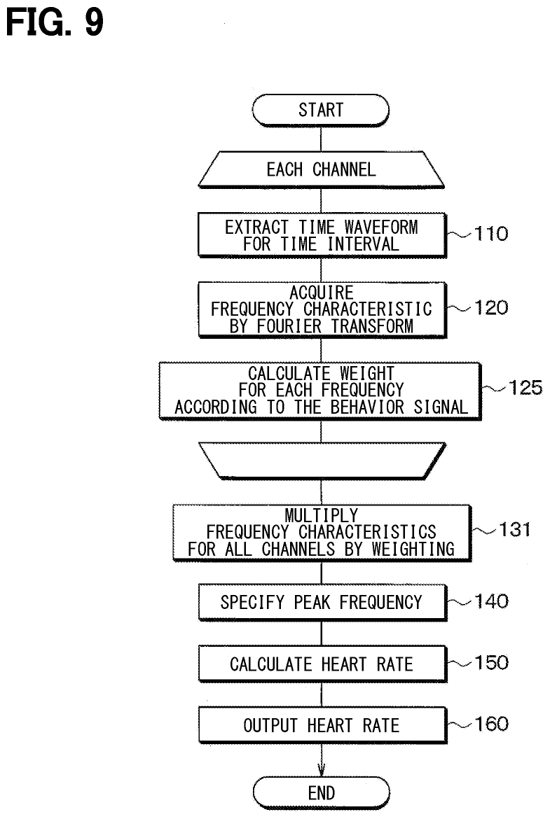

Next, a fifth embodiment will be described focusing on differences from the first embodiment. In this embodiment, as shown in FIG. 8, a vehicle speed sensor 7 and a gyro sensor 8 are added to the first embodiment. Further, the process of FIG. 9 executed by the processing unit 44 is replaced with the process of FIG. 9. Other than that, the configuration and operation of the present embodiment are the same as those of the first embodiment.

The vehicle speed sensor 7 outputs a pulse signal synchronized with the rotation of the wheels of the vehicle. It is possible to specify the traveling speed of the vehicle from the output interval of the pulse signal. The gyro sensor 8 outputs a signal according to the rotational angular velocity (for example, yaw rate) of the vehicle. The processing unit 44 acquires the signals output from the vehicle speed sensor 7 and the gyro sensor 8. In this way, the vehicle speed sensor 7 and the gyro sensor 8 both detect the traveling behavior of the vehicle and output a behavior signal corresponding to the traveling behavior.

Hereinafter, the contents of the process of FIG. 9 will be described. The steps with the same reference numerals in FIGS. 2 and 9 are the same except for the parts described below. The processing unit 44 acquires the frequency characteristics for each channel in the same manner as in the first embodiment in step 120, and then calculates the weight ω for each frequency according to the behavior signal in step 125. The behavior signal is one or both of the signal output from the vehicle speed sensor 7 and the signal output from the gyro sensor 8. The behavior signal includes noise derived from vibration generated in the vehicle during traveling.

Specifically, in step 125, the time waveform of the behavior signal acquired within a predetermined time interval is subjected to the discrete Fourier transform. As a result, the frequency characteristic indicating the relation between the frequency and the intensity of the behavior signal in the time interval is acquired. Then, the weight ω for each frequency is calculated according to the frequency characteristic of the behavior signal. Specifically, the value of the weight ω at each frequency becomes smaller as the intensity of the same frequency in the frequency characteristics of the behavior signal increases. Since such a weight ω has a small value at the frequency at which the vehicle vibrates, it can be used to reduce noise caused by the vibration of the vehicle.

After the repetition by the number of processing channels of steps 110, 120, and 125 is completed, the processing unit 44 proceeds to step 131. In step 131, the processing unit 44 multiplies the frequency characteristics obtained in step 120 for all channels by the weighting of the weight ω obtained in step 125 for all channels. Then, the relation between the frequency and the intensity obtained as a result of the weighted multiplication is defined as a synthetic frequency characteristic Q. This multiplication corresponds to a synthesis. The weighted multiplication method is the same as in step 131 of the second embodiment.

Subsequently, in step 140, the processing unit 44 specifies the peak frequency of the synthetic frequency characteristic Q obtained in the immediately preceding step 131, as in the first embodiment. Subsequently, in step 150, the heart rate of the heart 2a is specified based on the peak frequency specified in the immediately preceding step 140, as in the first embodiment. Subsequently, in step 160, the heart rate calculated in the immediately preceding step 150 is output to the output unit 43 as digital data. The output unit 43 outputs the digital data of the heart rate input from the processing unit 44 in this way to an external device outside of the biological information detection device 4.

As a result, the same effects as those of the first embodiment can be obtained. Further, the processing unit 44 synthesizes the frequency characteristics of the plurality of channels in a state where the weight ω corresponding to the traveling behavior of the vehicle is reflected, and obtains the synthetic frequency characteristic Q.

The first receiving antenna 13a and the second receiving antenna 13b are mounted on the vehicle. Therefore, most of the noise in the biological signals P1 and P2 is derived from the vibration generated according to the running behavior of the vehicle. In such a case, a synthetic frequency characteristic that reflects the weight according to the output from the vehicle behavior sensor (that is, the vehicle speed sensor 7, the gyro sensor 8) is used. Therefore, the S/N ratio of the synthetic frequency characteristic can be further improved.

Further, the vehicle behavior sensor includes one or both of the vehicle speed sensor 7 and the gyro sensor 8. The signals output from the vehicle speed sensor 7 and the gyro sensor 8 reflect the vibration applied to the vehicle during traveling. And the vibration applied to the vehicle tends to appear as noise in the biological signal. Therefore, the vehicle behavior sensor includes one or both of the vehicle speed sensor 7 and the gyro sensor 8. Thereby, the noise caused by the vibration of the vehicle can be effectively removed.

In the present embodiment, the processing unit 44 functions as a characteristic acquisition unit by executing step 120, functions as a synthesis unit by executing step 131, and functions as a calculation unit by executing step 150. Further, the processing unit 44 functions as a behavior weight calculation unit by executing step 125.

### Sixth Embodiment

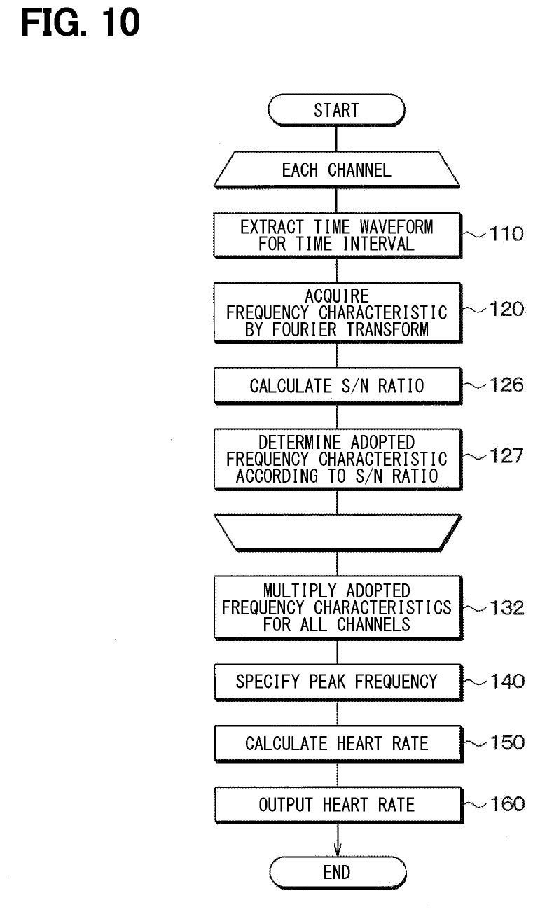

Next, a sixth embodiment will be described focusing on differences from the first embodiment. In the present embodiment, the process of FIG. 2 executed by the processing unit 44 with respect to the first embodiment is replaced with the process of FIG. 10. Other than that, the configuration and operation of the present embodiment are the same as those of the first embodiment.

Hereinafter, the contents of the process of FIG. 10 will be described. The steps with the same reference numerals in FIGS. 2 and 10 are the same except for the parts described below. The processing unit 44 acquires the frequency characteristics for each channel in the same manner as in the first embodiment in step 120, and then calculates the S/N ratio of the frequency characteristics in step 126. Specifically, the value obtained by dividing the intensity of the maximum peak in the frequency characteristic by the average value of the intensities at frequencies other than the peak is defined as the S/N ratio. Here, the maximum peak means the peak having the highest intensity.

Subsequently, in step 127, the processing unit 44 compares the S/N ratio calculated in the immediately preceding step 125 with a predetermined reference value. Then, if the S/N ratio is equal to or higher than the reference value, the frequency characteristic is adopted as a target for synthesis described later. However, if the S/N ratio is smaller than the reference value, the frequency characteristic is not adopted as a target for synthesis described later.

After the repetition by the number of processing channels of steps 110, 120, and 123 is completed, the processing unit 44 proceeds to step 132. In step 132, the processing unit 44 multiplies, by each other, the frequency characteristics determined to be adopted in step 127 among the frequency characteristics obtained in step 120 for all channels. The method of multiplication is the same as step 130 of the first embodiment.

In step 132, if there are two or more frequency characteristics determined to be adopted, multiplication is performed as described above. However, if there is one frequency characteristic determined to be adopted, the one frequency characteristic is regarded as the multiplication result. Then, the process is shifted to step 140.

Subsequently, in step 140, the processing unit 44 specifies the peak frequency of the synthetic frequency characteristic Q obtained in the immediately preceding step 132, as in the first embodiment. Subsequently, in step 150, the heart rate of the heart 2a is specified based on the peak frequency specified in the immediately preceding step 140, as in the first embodiment. Subsequently, in step 160, the heart rate calculated in the immediately preceding step 150 is output to the output unit 43 as digital data. The output unit 43 outputs the digital data of the heart rate input from the processing unit 44 in this way to an external device outside of the biological information detection device 4.

As a result, the same effects as those of the first embodiment can be obtained. Further, the processing unit 44 selects a frequency characteristic in which the S/N ratio of each of the plurality of frequency characteristics of the biological signals of the plurality of channels is equal to or higher than the reference value. The selected frequency characteristics are synthesized to obtain the synthetic frequency characteristics. In this way, the synthesized value is calculated by selecting the frequency characteristics that satisfy the condition that the S/N ratio is larger than the reference value. As a result, the calculation accuracy of biological information is improved.

In the present embodiment, the processing unit 44 functions as a characteristic acquisition unit by executing step 120, functions as a synthesis unit by executing step 132, and functions as a calculation unit by executing step 150. Further, the processing unit 44 functions as a selection unit by executing steps 126 and 127.

### Other Embodiments

The processing unit and method described in the present disclosure in the above embodiments may be implemented by one or more special-purpose computers, which may be created (i) by configuring (a) a memory and a processor programmed to execute one or more particular functions embodied in computer programs. Alternatively, the processing unit and method described in the present disclosure in the above embodiments may be implemented by one or more special-purpose computers, which may be created (ii) by configuring (b) a processor provided by one or more special-purpose hardware logic circuits. Furthermore, the processing unit and method described in the present disclosure in the above embodiments may be implemented by one or more special-purpose computers, which may be created (iii) by configuring a combination of (a) a memory and a processor programmed to execute one or more particular functions embodied in computer programs and (b) a processor provided by one or more special-purpose hardware logic circuits. The computer programs may be stored, as instructions to be executed by a computer, in a tangible non-transitory computer-readable storage medium.

The present disclosure is not limited to the above-described embodiments, and can be appropriately modified. The embodiments described above are not independent of each other, and can be appropriately combined except when the combination is obviously impossible. The constituent element(s) of each of the above embodiments is/are not necessarily essential unless it is specifically stated that the constituent element(s) is/are essential in the above embodiment, or unless the constituent element(s) is/are obviously essential in principle. Furthermore, in each of the above embodiments, in the case where the number of the constituent element(s), the value, the amount, the range, and/or the like is specified, the present disclosure is not necessarily limited to the number of the constituent element(s), the value, the amount, and/or the like specified in the embodiments unless the number of the constituent element(s), the value, the amount, and/or the like is indicated as indispensable or is obviously indispensable in view of the principle of the present disclosure. Further, in the above embodiment, when it is described that the external environment information of the vehicle (for example, the humidity outside the vehicle) is acquired from the sensor, the sensor may be abolished. It is also possible to receive the external environment information from a server or cloud outside the vehicle. It is also possible to acquire related information related to the external environment information from a server or cloud outside the vehicle and estimate the external environment information from the acquired related information. In particular, when a plurality of values are exemplified for a certain quantity, it is also possible to adopt a value between the plurality of values unless otherwise specified or when it is clearly impossible in principle. Further, in each of the embodiments described above, when referring to the shape, positional relation, and the like of the constituent elements and the like, it is not limited to the shape, positional relation, and the like, except for the case where the constituent elements are specifically specified, the case where the constituent elements are fundamentally limited to a specific shape, positional relation, and the like. Further, the present disclosure also allows the following modified examples and modified examples within a equivalent range for each of the above embodiments. In addition, the following modified examples can be independently selected to be applied or not applied to the above-described embodiments. That is, any combination of the following modified examples can be applied to the above embodiments.

### FIRST MODIFIED EXAMPLE

In the above embodiment, multiplication is disclosed as an example of synthesizing the frequency characteristics of a plurality of channels. However, the method for synthesizing the frequency characteristics of a plurality of channels is not limited to multiplication, and may be addition or any combination of multiplication and addition.

### SECOND MODIFIED EXAMPLE

In the above embodiments, the frequency characteristics of a plurality of channels are synthesized with each other at the same frequency. However, the above configuration is not necessarily required. For example, the frequency characteristics of a plurality of channels may be synthesized with a slight frequency shift.

### THIRD MODIFIED EXAMPLE

In step 121 of the second embodiment, the processing unit 44 calculates the time-course change amount in the intensity of the biological signal of the channel for each frequency, based on the frequency characteristics calculated in the immediately preceding step 120 in the present time interval and the frequency characteristics calculated in the previous time interval immediately before the present time interval. However, the above configuration is not necessarily required. For example, the processing unit 44 may calculate the time-course change amount in the intensity of the biological signal of the channel for each frequency, based on the frequency characteristics calculated in the immediately preceding step 120 in the present time interval and the frequency characteristics calculated in the second previous time interval or more earlier time interval before the present time interval. Further, for example, the processing unit 44 may calculate the time-course change amount in the intensity of the biological signal of the channel for each frequency based on the frequency characteristics calculated in three or more time intervals.

### FOURTH MODIFIED EXAMPLE

In step 121 of the second embodiment, the processing unit 44 calculates the time-course change amount in the intensity of the biological signal of the channel for each frequency based on the difference in frequency characteristics of the same channel in different time intervals. However, in step 121, the processing unit 44 may calculate the time-course change amount in the intensity of the biological signal of any of the different channels for each frequency based on the difference in the frequency characteristics of the different time intervals of the different channels.

### FIFTH MODIFIED EXAMPLE

In the fourth embodiment, in step 124, the processing unit 44 calculates a representative value of these two frequency characteristics based on the frequency characteristics acquired in the immediately preceding step 120 in the present time interval and the frequency characteristics of the previous time interval immediately before the present time interval. However, the above configuration is not necessarily required. For example, the processing unit 44 may calculate a representative value of these two frequency characteristics, based on the frequency characteristics calculated in the immediately preceding step 120 in the present time interval and the frequency characteristics calculated in the second previous time interval or more earlier time interval before the present time interval. The representative value of these two frequency characteristics may be calculated. Further, for example, the processing unit 44 may calculate a representative value of the three or more frequency characteristics based on the frequency characteristics calculated in the three or more time intervals.

### SIXTH MODIFIED EXAMPLE

In the fifth embodiment, the processing unit 44 has the same weight calculated in step 125 even if the channels are different. However, even when the used behavior signals are the same, the weight calculated in step 125 may be set to be different for each of the channels.

### SEVENTH MODIFIED EXAMPLE

In the second embodiment, the frequency has the smaller value of the weight ω as the frequency has the larger time-course change amount. However, conversely, the frequency may have the larger value of the weight ω as the frequency has the larger time-course change amount. In this case, in step 140, the processing unit 44 specifies the frequency having the lowest intensity among the peaks of the synthetic frequency characteristic Q obtained in the immediately preceding step 130 as a value for calculating the heart rate.

### EIGHTH MODIFIED EXAMPLE

In the above embodiment, the entire biological information detection system is mounted on the vehicle. However, part of the biometric information detection system does not have to be mounted on the vehicle. In that case, signals may be exchanged between the portion of the biological information detection system mounted on the vehicle and the portion not mounted on the vehicle by wireless communication or the like. Alternatively, the entire biometric information detection system may be installed outside the vehicle. That is, the biological information detection system may be used not only for calculating the biological information of the occupants of the vehicle but also for calculating the biological information of a person outside the vehicle (for example, inside a building).

### NINTH MODIFIED EXAMPLE

In the above embodiments, as the biological activity sensor, a radio wave type biological activity sensor, that is, the first receiving antenna 13a and the second receiving antenna 13b are exemplified. However, the biological activity sensor is not limited to such a sensor. For example, the biological activity sensor may be an ultrasonic sensor or a piezoelectric sensor embedded in a vehicle seat. Further, the biological activity sensor may be a non-contact type sensor such as these, or may not be a non-contact type sensor.

### TENTH MODIFIED EXAMPLE

The biological information calculated by the processing unit 44 in the above embodiment is a heart rate. However, the biological information calculated by the processing unit 44 does not have to be the heart rate. For example, the processing unit 44 may calculate the respiratory rate from the same biological signals P1 and P2. Alternatively, the processing unit 44 may calculate the pulse rate using another biological signal sensor. If the processing unit 44 calculates biological information regarding biological activities that are active in a substantially stable cycle, a technique such as the above embodiment is useful.

### ELEVENTH MODIFIED EXAMPLE

In the above embodiments, there are two biological activity sensors and, therefore, two channels. However, the number of biological activity sensors and the number of channels may be three or more. For example, in the sixth embodiment, suppose a case where the number of biological activity sensors and the number of channels are three or more. In this case, if there are two or more channels having an S/N ratio with the reference value or more, such two or more channels or the biological activity sensors can be selected and synthesized.

### TWELFTH MODIFIED EXAMPLE

In the third embodiment, the process of setting the heart rate statistic value as the weight ω is executed in step 123 of FIG. 6. This process of setting the heart rate statistic value as the weight ω may also be executed, in the second embodiment, before step 120 and after step 121 of the process of FIG. 4. In that case, the weight ω based on the heart rate statistic is calculated separately from the weight ω according to the change amount in intensity for each frequency. In that case, in step 131 of FIG. 4, both the weight ω based on the heart rate statistic value and the weight ω according to the change amount in intensity for each frequency are reflected in the synthesis.

### THIRTEENTH MODIFIED EXAMPLE

In the fourth embodiment, the process of calculating the representative value of the frequency characteristics of the plurality of time intervals is executed in step 124 of FIG. 7. This process of calculating the representative value may be executed immediately after step 120 of FIGS. 4 and 6 in the second and third embodiments. In that case, in step 131 of FIGS. 4 and 6, synthesis using the representative value is performed.

### FOURTEENTH MODIFIED EXAMPLE

In the fifth embodiment, the process of calculating the weight ω according to the behavior signal is executed in step 125 of FIG. 9. This process of calculating the weight ω according to the behavior signal may be executed immediately after the process step 120 of FIGS. 4, 6, and 7, in the second, third, and fourth embodiments. In that case, the weight ω according to the behavior signal is calculated separately from the weight ω of other types. In that case, in step 131 of FIGS. 4 and 6, all the calculated weights w are reflected in the synthesis. Further, in step 130 of FIG. 7, the weight ω corresponding to the behavior signal is reflected in the synthesis.

### FIFTEENTH MODIFIED EXAMPLE

The process in steps 126 and 127 of FIG. 10 in the sixth embodiment may be executed immediately after the process step 120 of FIGS. 4, 6, 7, and 9 in the second, third, fourth, and fifth embodiments. In that case, in the process of step 131 of FIGS. 4, 6 and 9, only the adopted frequency characteristic and the corresponding weight ω are used for the synthesis. Further, in the process of step 130 of FIG. 7, only the representative value of the adopted frequency characteristics are used for the synthesis.

For reference to further explain features of the present disclosure, the description is added as follows.

There is described a technology that subtracts a time waveform of the signal detected by a first piezoelectric element arranged near the seat mounting bracket from a time waveform of the signal detected by a second piezoelectric element embedded in the part of the backrest of the seat near the occupant's heart. Such a technology can remove vehicle noise included in the biological signal detected by the second piezoelectric element. Then, the technology calculates the heart rate of the passenger from the biological signal from which the vehicle noise is removed.

However, according to the study of the inventor, in the above technology, in addition to the sensor arranged at the position where the biological signal can be detected, it is necessary to arrange the sensor arranged at the position where the biological signal cannot be detected. Therefore, there is a technical difficulty in determining the position where the biological signal cannot be detected. Moreover, since the above-mentioned technology uses the difference between the time waveforms, there is a possibility that noise cannot be removed due to the influence of the phase shift of both signals. These things are the same even when calculating biological information other than heart rate.

It is thus desired to calculate biological information by suppressing the influence of phase shift and the influence of noise contained in the output of the sensor that detects biological signals by using a method different from the method of using a sensor placed in a position where biological signals cannot be detected.

Aspects of the present disclosure described herein are set forth in the following clauses.

According to a first aspect illustrated in part or all of the above embodiments, a biological information detection device is provided to include a characteristic acquisition unit (120, 124), a synthesis unit (130, 131, 132), and a calculation unit (150). The characteristic acquisition unit is configured to acquire a frequency characteristic (Q1, Q2) indicating a relation between a frequency and an intensity with respect to each of a plurality of biological signals (P1, P2) input respectively from a plurality of biological activity sensors (13a, 13b) arranged at a plurality of positions different from each other to detect a biological activity of a person (2). The synthesis unit is configured to obtain a synthetic frequency characteristic indicating the relation between the frequency and the intensity by synthesizing a plurality of frequency characteristics acquired by the characteristic acquisition unit from the plurality of biological signals. The calculation unit is configured to calculate biological information on the biological activity, based on the synthetic frequency characteristic (Q) obtained by the synthesis unit.

The present inventor has focused on the fact that the frequency characteristics of non-noise components of biological signals are generally stable. There is found a fact that if biological signals are detected by a plurality of biological activity sensors arranged at different positions, the frequency characteristics of noise contained in the biological signals detected by the plurality of biological activity sensors have a tendency to be significantly different. Thus the present inventor came up with the idea of using such a tendency.

That is, the frequency characteristics of the biological signals from a plurality of biological activity sensors arranged at different positions as described above are synthesized in the frequency domain. Thus the non-noise parts of the biological signal strengthen each other, and the noise parts do not strengthen each other. Therefore, the influence of noise is suppressed in the above-mentioned synthetic frequency characteristics obtained by synthesis. Moreover, since the frequency characteristics are synthesized, the phase shift does not affect the noise suppression.

Further according to a second aspect, the synthesis unit is configured to obtain the synthetic frequency characteristic by multiplying, by each other, the plurality of frequency characteristics acquired by the characteristic acquisition unit.

In this way, the S/N ratio of the synthetic frequency characteristic is improved by obtaining the synthetic frequency characteristic by multiplying the plurality of frequency characteristics by each other.

Further, according to a third aspect, the synthesis unit is further configured to obtain the synthetic frequency characteristic by synthesizing, with respect to each of frequencies, the plurality of frequency characteristics acquired by the characteristic acquisition unit from the plurality of biological signals with each other.

In this way, by synthesizing a plurality of frequency characteristics with the same frequency to obtain the synthetic frequency characteristic, the S/N ratio of the synthetic frequency characteristics is improved.

Further according to a fourth aspect, the biological information detection device further includes a change weight calculation unit (121). Herein, with respect to each of the plurality of biological signals input respectively from the plurality of biological activity sensors in a predetermined time interval, the characteristic acquisition unit is further configured to acquire the frequency characteristic indicating the relation between the frequency and the intensity in the time interval by converting a time waveform indicating a time-course change in the intensity in the time interval. Further, with respect to the frequency characteristic in the time interval of each of the plurality of biological signals, the change weight calculation unit is configured to calculate a time-course change amount in the intensity specific to each of frequencies based on the frequency characteristic indicating the relation between the frequency and the intensity in a period other than the time interval of each of the plurality of biological signals, and calculate a weight specific to each of frequencies according to the calculated time-course change amount. Further, the synthesis unit is configured to obtain the synthetic frequency characteristic by synthesizing the plurality of frequency characteristics acquired by the characteristic acquisition unit from the plurality of biological signals in a state of reflecting a plurality of weights calculated by the change weight calculation unit respectively corresponding to the plurality of biological signals.

As described above, with respect to the frequency characteristics calculated in the predetermined time interval, the weight for each frequency is determined based on the time-course change amount for each frequency based on the frequency characteristic in the period other than the time interval. As described above, the frequency characteristic of noise tends to differ greatly depending on the location where the biological activity sensor is installed, but also tends to differ greatly depending on the difference in the acquisition period of the biological signal. On the other hand, the frequency characteristic of the non-noise component of the biological signal is generally stable over time. Focusing on these points, the inventor came up with the idea that frequencies whose intensities fluctuate significantly over time are considered to be derived from noise.

For that purpose, as described above, the biological information detection device reflects the weight for each frequency in the synthetic frequency characteristic according to the time-course change amount in intensity for each frequency based on the frequency characteristic in a period other than the predetermined time interval. Thereby, the S/N ratio of the synthetic frequency characteristic can be further improved by utilizing the characteristic of the biological signal in the frequency domain.

Further according to a fifth aspect, the weight specific to one of frequencies is smaller as an absolute value of the time-course change amount specific to the one of frequencies is larger. By doing so, the weight can be set as a more intuitive quantity.

Further according to a sixth aspect, with respect to each of the plurality of biological signals input respectively from the plurality of biological activity sensors in each of a plurality of time intervals, the characteristic acquisition unit is further configured to acquire the frequency characteristic indicating the relation between the frequency and the intensity in each of the plurality of time intervals by converting a time waveform indicating a time-course change in the intensity in each of the plurality of time intervals. Further, with respect to each of the plurality of biological signals, the characteristic acquisition unit is further configured to calculate a representative value of the frequency characteristics in the plurality of time intervals. Further, the synthesis unit is further configured to obtain the synthetic frequency characteristic by synthesizing a plurality of representative values of the plurality of biological signals calculated by the characteristic acquisition unit.

In this way, by obtaining the synthetic frequency characteristic using the representative value of two or more time intervals, the S/N ratio of the synthetic frequency characteristic is improved.

Further, according to a seventh aspect, the biological information detection device further includes a behavior weight calculation unit (125). Herein, the plurality of biological activity sensors are mounted on a vehicle that is provided with a vehicle behavior sensor (7, 8) to output a behavior signal according to a traveling behavior of the vehicle. Further, the behavior weight calculation unit is configured to calculate a weight according to the behavior signal with respect to the frequency characteristic of each of the plurality of biological signals. Further, the synthesis unit is configured to obtain the synthetic frequency characteristic by synthesizing the plurality of frequency characteristics acquired by the characteristic acquisition unit from the plurality of biological signals in a state of reflecting a plurality of weights calculated by the behavior weight calculation unit with respect to the plurality of biological signals.

When the biological activity sensor is mounted on the vehicle, the noise in the biological signal is often derived from the running behavior of the vehicle. In such a case, the S/N ratio of the synthetic frequency characteristic can be further improved by using the synthetic frequency characteristic that reflects the weight corresponding to the output from the vehicle behavior sensor.

Further, according to an eighth aspect, the vehicle behavior sensor includes a vehicle speed sensor. The signal output from the vehicle speed sensor reflects the vibration applied to the vehicle during traveling. And the vibration applied to the vehicle tends to appear as noise in the biological signal. Therefore, when the vehicle behavior sensor includes the vehicle speed sensor, noise caused by the vibration of the vehicle can be effectively removed.

Further, according to a ninth aspect, the vehicle behavior sensor includes a gyro sensor. The signal output from the gyro sensor reflects the vibration applied to the vehicle during traveling. And the vibration applied to the vehicle tends to appear as noise in the biological signal. Therefore, when the vehicle behavior sensor includes the gyro sensor, noise caused by the vibration of the vehicle can be effectively removed.

Further, according to a tenth aspect, the biological information detection device further includes a selection unit (126, 127) configured to select the frequency characteristics each having an S/N ratio equal to or higher than a reference value among the plurality of frequency characteristics of the plurality of biological signals acquired by the characteristic acquisition unit. Herein, the synthesis unit is configured to obtain the synthetic frequency characteristic by synthesizing the frequency characteristics selected by the selection unit. As described above, by selecting the frequency characteristic satisfying the condition that the S/N ratio is larger than the reference value and calculating the synthetic value, the calculation accuracy of the biological information is improved.

What is claimed is:

1. A biological information detection device comprising:
a characteristic acquisition unit configured to acquire a frequency characteristic indicating a relation between a frequency and an intensity with respect to each of a plurality of biological signals input respectively from a plurality of biological activity sensors arranged at a plurality of positions different from each other to detect a biological activity of a person;
a synthesis unit configured to obtain a synthetic frequency characteristic indicating the relation between the frequency and the intensity by synthesizing a plurality of frequency characteristics acquired by the characteristic acquisition unit from the plurality of biological signals;
a calculation unit configured to calculate biological information on the biological activity, based on the synthetic frequency characteristic obtained by the synthesis unit; and
a change weight calculation unit,
wherein:
with respect to each of the plurality of biological signals input respectively from the plurality of biological activity sensors in a predetermined time interval, the characteristic acquisition unit is further configured to acquire the frequency characteristic indicating the relation between the frequency and the intensity in the time interval by converting a time waveform indicating a time-course change in the intensity in the time interval;
with respect to the frequency characteristic in the time interval of each of the plurality of biological signals, the change weight calculation unit is configured to calculate a time-course change amount in the intensity specific to each of frequencies based on the frequency characteristic indicating the relation between the frequency and the intensity in a period other than the time interval of each of the plurality of biological signals, and calculate a weight specific to each of frequencies according to the calculated time-course change amount; and
the synthesis unit is configured to obtain the synthetic frequency characteristic by synthesizing the plurality of frequency characteristics acquired by the characteristic acquisition unit from the plurality of biological signals in a state of reflecting a plurality of weights calculated by the change weight calculation unit respectively corresponding to the plurality of biological signals.

2. The biological information detection device according to claim 1, wherein:
the weight specific to one of frequencies is smaller as an absolute value of the time-course change amount specific to the one of frequencies is larger.

3. The biological information detection device according to claim 1, wherein:
the synthesis unit is configured to obtain the synthetic frequency characteristic by multiplying, by each other, the plurality of frequency characteristics acquired by the characteristic acquisition unit.

4. The biological information detection device according to claim 1, wherein:
the synthesis unit is further configured to obtain the synthetic frequency characteristic by synthesizing, with respect to each of frequencies, the plurality of frequency characteristics acquired by the characteristic acquisition unit from the plurality of biological signals with each other.

5. The biological information detection device according to claim 1, wherein:
with respect to each of the plurality of biological signals input respectively from the plurality of biological activity sensors in each of a plurality of time intervals, the characteristic acquisition unit is further configured to acquire the frequency characteristic indicating the relation between the frequency and the intensity in each of the plurality of time intervals by converting a time waveform indicating a time-course change in the intensity in each of the plurality of time intervals;
with respect to each of the plurality of biological signals, the characteristic acquisition unit is further configured to calculate a representative value of the frequency characteristics in the plurality of time intervals; and
the synthesis unit is further configured to obtain the synthetic frequency characteristic by synthesizing a plurality of representative values of the plurality of biological signals calculated by the characteristic acquisition unit.

6. The biological information detection device according to claim 1, further comprising:
a selection unit configured to select the frequency characteristics each having an S/N ratio equal to or higher than a reference value among the plurality of frequency characteristics of the plurality of biological signals acquired by the characteristic acquisition unit,
wherein:
the synthesis unit is configured to obtain the synthetic frequency characteristic by synthesizing the frequency characteristics selected by the selection unit.

7. The biological information detection device according to claim 1, further comprising:
a behavior weight calculation unit,
wherein:
the plurality of biological activity sensors are mounted on a vehicle that is provided with a vehicle behavior sensor to output a behavior signal according to a traveling behavior of the vehicle;
the behavior weight calculation unit is configured to calculate a weight according to the behavior signal with respect to the frequency characteristic of each of the plurality of biological signals; and
the synthesis unit is configured to obtain the synthetic frequency characteristic by synthesizing the plurality of frequency characteristics acquired by the characteristic acquisition unit from the plurality of biological signals in a state of reflecting a plurality of weights calculated by the behavior weight calculation unit with respect to the plurality of biological signals.

8. The biological information detection device according to claim 7, wherein:
the vehicle behavior sensor includes a vehicle speed sensor.

9. The biological information detection device according to claim 7, wherein:
the vehicle behavior sensor includes a gyro sensor.

10. A biological information detection device comprising:
a characteristic acquisition unit configured to acquire a frequency characteristic indicating a relation between a frequency and an intensity with respect to each of a plurality of biological signals input respectively from a plurality of biological activity sensors arranged at a plurality of positions different from each other to detect a biological activity of a person;
a synthesis unit configured to obtain a synthetic frequency characteristic indicating the relation between the frequency and the intensity by synthesizing a plurality of frequency characteristics acquired by the characteristic acquisition unit from the plurality of biological signals;
a calculation unit configured to calculate biological information on the biological activity, based on the synthetic frequency characteristic obtained by the synthesis unit; and
a behavior weight calculation unit,
wherein:
the plurality of biological activity sensors are mounted on a vehicle that is provided with a vehicle behavior sensor to output a behavior signal according to a traveling behavior of the vehicle;
the behavior weight calculation unit is configured to calculate a weight according to the behavior signal with respect to the frequency characteristic of each of the plurality of biological signals; and
the synthesis unit is configured to obtain the synthetic frequency characteristic by synthesizing the plurality of frequency characteristics acquired by the characteristic acquisition unit from the plurality of biological signals in a state of reflecting a plurality of weights calculated by the behavior weight calculation unit with respect to the plurality of biological signals.

11. The biological information detection device according to claim 10, wherein:
the vehicle behavior sensor includes a vehicle speed sensor.

12. The biological information detection device according to claim 10, wherein:
the vehicle behavior sensor includes a gyro sensor.

13. The biological information detection device according to claim 10, wherein:
the synthesis unit is configured to obtain the synthetic frequency characteristic by multiplying, by each other, the plurality of frequency characteristics acquired by the characteristic acquisition unit.

14. The biological information detection device according to claim 10, wherein:
the synthesis unit is further configured to obtain the synthetic frequency characteristic by synthesizing, with respect to each of frequencies, the plurality of frequency characteristics acquired by the characteristic acquisition unit from the plurality of biological signals with each other.

15. The biological information detection device according to claim 10, wherein:
with respect to each of the plurality of biological signals input respectively from the plurality of biological activity sensors in each of a plurality of time intervals, the characteristic acquisition unit is further configured to acquire the frequency characteristic indicating the relation between the frequency and the intensity in each of the plurality of time intervals by converting a time waveform indicating a time-course change in the intensity in each of the plurality of time intervals;
with respect to each of the plurality of biological signals, the characteristic acquisition unit is further configured to calculate a representative value of the frequency characteristics in the plurality of time intervals; and
the synthesis unit is further configured to obtain the synthetic frequency characteristic by synthesizing a plurality of representative values of the plurality of biological signals calculated by the characteristic acquisition unit.

16. The biological information detection device according to claim 10, further comprising:
a selection unit configured to select the frequency characteristics each having an S/N ratio equal to or higher than a reference value among the plurality of frequency characteristics of the plurality of biological signals acquired by the characteristic acquisition unit,
wherein:
the synthesis unit is configured to obtain the synthetic frequency characteristic by synthesizing the frequency characteristics selected by the selection unit.

17. The biological information detection device according to claim 10, further comprising:
a change weight calculation unit,
wherein:
with respect to each of the plurality of biological signals input respectively from the plurality of biological activity sensors in a predetermined time interval, the characteristic acquisition unit is further configured to acquire the frequency characteristic indicating the relation between the frequency and the intensity in the time interval by converting a time waveform indicating a time-course change in the intensity in the time interval;
with respect to the frequency characteristic in the time interval of each of the plurality of biological signals, the change weight calculation unit is configured to calculate a time-course change amount in the intensity specific to each of frequencies based on the frequency characteristic indicating the relation between the frequency and the intensity in a period other than the time interval of each of the plurality of biological signals, and calculate a weight specific to each of frequencies according to the calculated time-course change amount; and
the synthesis unit is configured to obtain the synthetic frequency characteristic by synthesizing the plurality of frequency characteristics acquired by the characteristic acquisition unit from the plurality of biological signals in a state of reflecting a plurality of weights calculated by the change weight calculation unit respectively corresponding to the plurality of biological signals.

18. The biological information detection device according to claim 17, wherein:
the weight specific to one of frequencies is smaller as an absolute value of the time-course change amount specific to the one of frequencies is larger.

19. The biological information detection device according to claim 1, further comprising:
one or more than one processor communicably coupled to the plurality of biological activity sensors arranged at the plurality of positions different from each other, the processor being configured to implement the characteristic acquisition unit, the synthesis unit, the calculation unit, and the change weight calculation unit.

20. The biological information detection device according to claim 10, further comprising:
one or more than one processor communicably coupled to the plurality of biological activity sensors arranged at the plurality of positions different from each other, the processor being configured to implement the characteristic acquisition unit, the synthesis unit, the calculation unit, and the behavior weight calculation unit.

21. A biological information detection device comprising:
one or more than one processor communicably coupled to a plurality of biological activity sensors arranged at a plurality of positions different from each other to detect a biological activity of a person, the processor being configured to:
acquire a plurality of frequency characteristics respectively corresponding to a plurality of biological signals input respectively from the plurality of biological activity sensors, each of the plurality of frequency characteristic indicating a relation between a frequency and an intensity with respect to each of the plurality of biological signals;
obtain a synthetic frequency characteristic indicating the relation between the frequency and the intensity by synthesizing the plurality of frequency characteristics acquired from the plurality of biological signals;
calculate biological information on the biological activity, based on the synthetic frequency characteristic,
wherein:
with respect to each of the plurality of biological signals input respectively from the plurality of biological activity sensors in a predetermined time interval, the processor is further configured to acquire each of the frequency characteristics indicating the relation between the frequency and the intensity in the time interval by converting a time waveform indicating a time-course change in the intensity in the time interval;
with respect to each of the plurality of frequency characteristics in the time interval of each of the plurality of biological signals, the processor is further configured to calculate a time-course change amount in the intensity specific to each of frequencies based on each of the plurality of frequency characteristics indicating the relation between the frequency and the intensity in a period other than the time interval of each of the plurality of biological signals, and calculate a plurality of weights each of which is specific to each of frequencies according to the calculated time-course change amount; and
the processor is further configured to obtain the synthetic frequency characteristic by synthesizing the plurality of frequency characteristics acquired from the plurality of biological signals in a state of reflecting the plurality of weights respectively corresponding to the plurality of biological signals.
